(19) United States
(12) Patent Application Publication
(10) Pub. No.: US 2010/0249606 A1
(43) Pub. Date: Sep. 30, 2010

(54) METHOD AND DEVICE FOR RECONSTRUCTING A FLUORESCENCE IMAGE OF THE INTERIOR OF A TURBID MEDIUM

(75) Inventors: RONNY ZIEGLER, FURTH (DE); TIM NIELSEN, HAMBURG (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/728,690

(22) Filed: Mar. 22, 2010

(30) Foreign Application Priority Data
Mar. 24, 2009 (EP) 09155952.6

(51) Int. Cl. A61B 6/00

(52) U.S. Cl. 600/473

(57) ABSTRACT

A method for reconstructing a fluorescence image of the interior of a turbid medium is provided. Initial Green's functions for the light propagation in the turbid medium for irradiation light are calculated from the diffusion equation based on an initial assumption for the optical properties of the turbid medium. Optical properties are reconstructed as a function of the position in the interior of the turbid medium based on the results of an attenuation measurement. Updated Green's functions for the light propagation in the turbid medium for irradiation light are calculated from the diffusion equation based on the reconstructed optical properties of the turbid medium. Updated Green's functions for the light propagation in the turbid medium for fluorescence light are calculated from the diffusion equation based on the reconstructed optical properties of the turbid medium and based on an assumed contrast agent distribution. The spatial distribution of a fluorescent contrast agent is reconstructed based on the results of the fluorescence measurement. Updated Green's functions for the light propagation in the turbid medium for fluorescence light are calculated from the diffusion equation based on the reconstructed optical properties of the turbid medium and based on the reconstructed spatial distribution of the contrast agent. An updated spatial distribution of the fluorescent contrast agent is reconstructed based on the results of the fluorescence measurement.

### FIELD OF INVENTION

The present invention relates to a method for reconstructing a fluorescence image of the interior of a turbid medium and to a device for reconstructing a fluorescence image of the interior of a turbid medium.

### BACKGROUND OF THE INVENTION

In the context of the present application, the term turbid medium is to be understood to mean a substance consisting of a material having a high light scattering coefficient, such as for example intralipid solution or biological tissue. Further, light is to be understood to mean electromagnetic radiation, in particular electromagnetic radiation having a wavelength in the range from 180 nm to 1400 nm. The term “optical properties” covers the wavelength dependent reduced scattering coefficient μ′ₛ, the absorption coefficient μₐ, and the diffusion coefficient D.

A method for imaging the interior of turbid media, e.g. for breast cancer screening, which has become popular in recent years is imaging by use of light, in particular using light in the near infrared (NIR). Such methods are implemented in mammography devices and devices for examining other parts of human or animal bodies. A prominent example for such a method for imaging the interior of a turbid medium by means of light is Diffuse Optical Tomography (DOT). For example, such a DOT device for imaging the interior of a turbid medium uses a light source to irradiate the turbid medium and photodetectors for measuring a part of the light transported through the turbid medium, i.e. its intensity. A control unit is provided for controlling the scanning process. A processing unit is provided for reconstructing an image of the interior of the turbid medium on the basis of the measured intensities. Some of the known devices are particularly adapted for examining female breasts. In order to allow the examination of the turbid medium, the device is provided with a receiving portion enclosing a measurement volume and arranged to receive the turbid medium. Light from the light source is coupled into the receiving volume and into the turbid medium. The light is chosen such that it is capable of propagating through the turbid medium. For imaging an interior of a female breast, light in the NIR (near infrared) is typically used. Scattered light emanating from the turbid medium as a result of coupling light into the receiving volume is coupled out of the receiving volume. Light coupled out of the receiving volume is detected and used to reconstruct an image of an interior of the turbid medium. Due to different sizes of the turbid media to be examined, the size of the receiving portion may not perfectly match the size of the turbid medium, i.e. a space remains between the boundary of the receiving volume and the turbid medium. The part of the turbid medium under investigation can be surrounded by a scattering medium (coupling medium) filling the space in the receiving volume. The scattering medium is chosen such that the optical parameters of the scattering medium, such as the absorption and scattering coefficients, are similar to the corresponding optical parameters of the turbid medium. The light source subsequently irradiates the turbid medium from different directions and the photodetectors measure a part of the light transmitted through the turbid medium. A plurality of such measurements are performed with the light directed to the turbid medium from different directions and, based on the results of the measurements, i.e. the obtained data set, the processing unit reconstructs the image of the examined turbid medium.

According to one development of this method, attenuation scans for light are performed in which the attenuation of light is detected for a plurality of combinations of source positions and detection positions. In these measurements the intrinsic contrast of the turbid medium is used, i.e. light at different wavelengths is attenuated by different amounts due to the presence of scatterers and chromophores such as oxy-hemoglobin, deoxy-hemoglobin, water, and lipids. From these attenuation scans, absorption and scattering images of the turbid medium can be reconstructed as well as images of physiological parameters such as e.g. the hemoglobin concentration. This technology has become known as Diffuse Optical Tomography (DOT).

According to a further development of this method, a fluorescent contrast agent which preferentially accumulates in lesions in the turbid medium under investigation, e.g. cancerous tissue in a female breast, is administered for the measurement. The turbid medium is irradiated with light from a light source, preferably a laser, and the fluorescent light which is emitted by the turbid medium is detected in addition to the light from the light source. From this measurement, a volumetric image of the contrast agent distribution in the breast is reconstructed, i.e. exogenous contrast is used. Thus, the spatial distribution of the contrast agent in the turbid medium is reconstructed. This method is called Diffuse Optical Fluorescence Tomography.

Devices have been developed which are adapted to perform both attenuation measurements and fluorescence measurements. In such devices, typically attenuation measurements are performed for a plurality of wavelengths in a certain range of wavelengths. The data obtained in the attenuation measurements allows reconstructing a spatially resolved image of the absorption properties of the turbid medium for the plurality of wavelengths. Thus, it can be reconstructed from the attenuation measurements how the light travels through the turbid medium at these wavelengths. In order to perform fluorescence measurements, the fluorescent contrast agent is explicitly excited and solely the light emitted by the fluorescent contrast agent is measured. This is for example achieved by introducing appropriate filters in the light paths between the measurement volume and a detection unit. The fluorescent contrast agent administered to the turbid medium under examination influences the propagation of light in the turbid medium, since it absorbs the light from the light source. This effect becomes particularly relevant at higher contrast agent concentrations. However, this effect is not taken into account in known reconstruction algorithms used to reconstruct an image of the interior of the turbid medium based on the detected light. Consequently, a reconstructed image may comprise errors which are due to the light absorption by the fluorescent contrast agent.

### SUMMARY OF THE INVENTION

It is an object of the present invention to improve the quality of reconstructed images of the interior of turbid media.

This object is solved by a method for reconstructing a fluorescence image of the interior of a turbid medium according to claim 1. The method comprises the steps: accommodating a turbid medium to which a fluorescent contrast agent has been administered in a measurement volume, performing an attenuation measurement by irradiating the turbid medium with irradiation light and detecting light emanating from the turbid medium; performing a fluorescence measurement by irradiating the turbid medium with light causing the fluorescent contrast agent to emit fluorescence light and detecting the fluorescence light emanating from the turbid medium. The method further comprises the steps: i) calculating initial Green's functions for the light propagation in the turbid medium for irradiation light from the diffusion equation based on an initial assumption for the optical properties of the turbid medium; ii) reconstructing optical properties as a function of the position in the interior of the turbid medium based on the results of the attenuation measurements; iii) calculating updated Green's functions for the light propagation in the turbid medium for irradiation light from the diffusion equation based on the reconstructed optical properties of the turbid medium; iv) calculating updated Green's functions for the light propagation in the turbid medium for the fluorescence light from the diffusion equation; v) reconstructing the spatial distribution of the fluorescent contrast agent based on the results of the fluorescence measurement; vi) recalculating updated Green's functions for the light propagation in the turbid medium for fluorescence light from the diffusion equation based on the reconstructed optical properties of the turbid medium and based on the reconstructed spatial distribution of the contrast agent; and vii) reconstructing an updated spatial distribution of the fluorescent contrast agent based on the results of the fluorescence measurement. Thus, according to the method for reconstructing a fluorescence image of the interior of a turbid medium, the reconstructed spatial distribution of the fluorescent contrast agent is fed back to the step of calculating the Green's functions for light propagation in the turbid medium and an updated spatial distribution of the fluorescent contrast agent is reconstructed based on the thus-updated Green's functions. As a consequence, the spatial distribution of the fluorescent contrast agent is finally reconstructed with improved quality even in the case of large contrast agent concentrations since self-absorption is taken into account for.

Preferably, the results of the attenuation measurement and the results of the fluorescence measurement each comprise values for a plurality of different light paths through the interior of the measurement volume. This can e.g. be achieved by subsequently irradiating the measurement volume with light from a plurality of different source positions and, for each source position, detecting light emanating from the measurement volume in a plurality of detection positions. In this case, a large plurality of detected values for different combinations of source position and detection position is provided. However, alternative arrangements realizing e.g. only one source position and a plurality of detection positions or a plurality of source positions and only one detection position are possible as well. Different source positions and detection positions need not necessarily be fixed with respect to the measurement volume. It is known, for instance, to rotate a detector unit during a measurement to assume different detection positions or to rotate a source unit to assume different source positions.

Preferably, in step ii), the optical properties of the turbid medium are reconstructed based on the results of the attenuation measurement and on the results of a reference measurement with the measurement volume filled with a reference medium. In this case, by exploiting the results of the reference measurement, the results of the attenuation measurement can be easily normalized, e.g. by dividing the measured results from the attenuation measurement by the corresponding results from the reference measurement.

Preferably, in steps v) and vii), the spatial distribution of the fluorescent contrast agent inside the turbid medium is reconstructed based on the results of the fluorescence measurement and on the results of the attenuation measurement.

If, in step vii), the updated spatial distribution of the fluorescent contrast agent inside the turbid medium is reconstructed exploiting the updated Green's functions for the light propagation in the turbid medium for fluorescence light recalculated in step vi), the reconstructed spatial distribution of the fluorescent contrast agent is advantageously fed back as an input for a new reconstruction of the fluorescent contrast agent distribution. Thus, an improved image of the spatial distribution of the contrast agent is reliably achieved even for high concentrations of the contrast agent.

If steps vi) and vii) are repeated an iterative process is realized which optimizes the reconstructed fluorescence image. The iteration is stopped when a convergence criterion is met. There are several known convergence criteria, e.g. stopping if the change of the reconstructed fluorescence image between two steps of the iteration is negligible.

Preferably, attenuation measurements are performed for a plurality of different wavelengths of the irradiation light, and, in step ii), the wavelength dependency of the optical properties is described by a model and the spatial distribution of the model parameters is reconstructed based on the results of the attenuation measurements at the plurality of different wavelengths, and optical properties as a function of the wavelength and position in the interior of the turbid medium are calculated based on the reconstructed spatial distribution of the model parameters. In this case, since a plurality of attenuation measurements at different wavelengths are performed, more information for image reconstruction is available and the image can be reconstructed with even higher accuracy.

If the optical properties are individually calculated for irradiation light and for fluorescence light, the Green's functions used for reconstructing the spatial distribution of the fluorescent contrast agent provide improved information resulting in improved fluorescence image quality.

The algorithmic steps described above can be carried out for measurement data obtained from continuous wave (cw) light sources as well as pulsed or modulated light sources. It is advantageous to used modulated or pulsed light sources because these measurements provide more information than cw measurements. For example, not only the light intensity is measured but also values for demodulation and phase shift or the temporal point spread function are obtained for modulated or pulsed measurements, respectively.

The object is also solved by a device for reconstructing a fluorescence image of the interior of a turbid medium according to claim 9. The device comprises: a measurement volume adapted for receiving a turbid medium to be examined; a light source unit adapted for irradiating the interior of the measurement volume; a detection unit adapted for detecting light emanating from the interior of the measurement volume; the light source unit and the detection unit being adapted such that a plurality of combinations of source position and detection position corresponding to a plurality of different light paths through the measurement volume are provided; and a control and reconstruction unit. The control and reconstruction unit is adapted to control the device to: perform an attenuation measurement by irradiating the turbid medium with irradiation light and detecting light emanating from the turbid medium; perform a fluorescence measurement by irradiating the turbid medium with light causing a fluorescent contrast agent administered to the turbid medium to emit fluorescence light and detecting the fluorescence light emanating from the turbid medium. The control and reconstruction unit is adapted to: i) calculate initial Green's functions for the light propagation in the turbid medium for irradiation light from the diffusion equation based on an initial assumption for the optical properties of the turbid medium; ii) reconstruct optical properties as a function of the position in the interior of the turbid medium based on the results of the attenuation measurements; iii) calculate updated Green's functions for the light propagation in the turbid medium for irradiation light from the diffusion equation based on the reconstructed optical properties of the turbid medium; iv) calculate updated Green's functions for the light propagation in the turbid medium for the fluorescence light from the diffusion equation; v) reconstruct the spatial distribution of the fluorescent contrast agent based on the results of the fluorescence measurement; vi) recalculate updated Green's functions for the light propagation in the turbid medium for fluorescence light from the diffusion equation based on the reconstructed optical properties of the turbid medium and based on the reconstructed spatial distribution of the contrast agent; and vii) reconstruct an updated spatial distribution of the fluorescent contrast agent based on the results of the fluorescence measurement. Thus, the reconstructed spatial distribution of the fluorescent contrast agent is fed back to the step in which Green's functions are calculated. In doing so, the spatially resolved fluorescent contrast agent concentration can be iteratively recalculated again and again, each time with improved results.

Preferably, the light source unit is adapted to selectively irradiate the interior of the measurement volume with light at a plurality of different wavelengths. In this case, attenuation measurements can be performed at different wavelengths such that the concentrations of different constituents of the turbid medium can be reconstructed from the attenuation measurements in a spatially resolved manner.

Preferably, the control and reconstruction unit is adapted to iteratively repeat steps vi) and vii) such that improved results for a reconstructed spatial distribution of the fluorescent contrast agent are iteratively achieved.

Preferably, the device is a medical image acquisition device.

### BRIEF DESCRIPTION OF THE DRAWINGS

Further features and advantages of the present invention will arise from the detailed description of embodiments with reference to the enclosed drawings.

FIG. 1 schematically shows a measurement volume of a device for imaging the interior of a turbid medium.

FIG. 2 schematically shows an arrangement of the measurement volume, the light source unit, and the detection unit in the device of FIG. 1.

FIG. 3 schematically shows different steps of a method according to the embodiment.

### DETAILED DESCRIPTION OF EMBODIMENTS

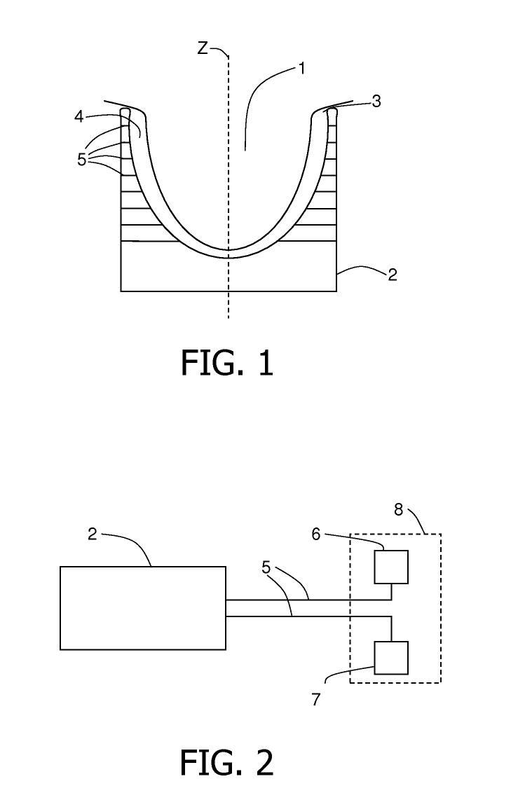

An embodiment of the present invention will now be described with reference to FIGS. 1 and 2. The device for reconstructing a fluorescence image of the interior of a turbid medium according to the embodiment is a device for diffuse optical tomography (DOT). In particular, the device is adapted for examination of female breasts. The overall construction of such a device is known in the art. The device comprises a bed (not shown) on which the person under examination is lying in a prone position. An opening is formed in the bed below which a measurement volume 4 extends. The measurement volume 4 is shown in FIG. 1.

In the device shown in FIG. 1, the turbid medium 1 to be examined is a female human breast. The measurement volume 4 is bounded by a receiving portion 2 adapted to receive the turbid medium 1, as schematically indicated in FIG. 1. The receiving portion 2 has a cup-like shape and is provided with an opening 3. As can be seen in FIG. 1, the turbid medium 1 to be examined is placed in the measurement volume 4 such that it freely hangs in the measurement volume 4 from the side of the opening 3. The receiving portion 2 serves to position and stabilize the turbid medium 1 which is examined.

The inner surface of the receiving portion 2 facing the turbid medium 3 is provided with a plurality of ends of light guides 5 formed by optically guiding fibers connecting to a light source unit 6 and to a detection unit 7, as schematically shown in FIG. 2.

The light source unit 6 comprises at least one light source capable of emitting light, preferably laser light. Preferably, the light source unit 6 is adapted for selectively emitting light of a plurality of different wavelengths. Preferably the light source unit 6 comprises a plurality of lasers each emitting monochromatic light but at different wavelengths. In this case, the light source unit 6 is adapted such that light from one of the light sources can be selectively coupled into the measurement volume at a time. However, it should be noted that it is also possible that the light source unit 6 is only capable of emitting light of one specific wavelength or band of wavelengths.

The ends of the light guides 5 are distributed on the inner surface of the receiving portion 2. The device is adapted such that light from the light source unit 6 can be directed to the turbid medium 1 from a plurality of different directions (source positions) and light emanating from the turbid medium 1 can be detected by the detection unit 7 in a plurality of different detection positions distributed around the measurement volume 4. In the embodiment, the detection unit 7 is implemented by a plurality of detectors the corresponding light guides 5 of which are distributed on the inner surface of the receiving portion 2. The ends of the light guides 5 at the inner surface of the receiving portion 2 form a plurality of source positions and a plurality of detection positions. In the embodiment the overall number of source positions is equal to the overall number of detection positions; however, the invention is not limited to an equal number. For example, in the device according to the embodiment, 256 different source positions are provided and 256 detection positions, i.e. respective ends of light guides 5 are provided on the inner surface of the receiving portion 2. The light from the light source is subsequently directed to the turbid medium 1 from the 256 source positions and, for each source position, the light emanating from the turbid medium 1 is detected in the 256 detection positions. However, the invention is not limited to these specific numbers.

As schematically shown in FIG. 2, the device comprises a control and processing unit 8 for controlling the acquisition of images and reconstructing images of the interior of the turbid medium 1. The control and processing unit 8 reconstructs an image of the interior of the turbid medium 1 based on the signals from the detection unit 7. For reconstruction, the signals sampled during a scan in which the light is directed to the turbid medium 1 from different directions are used. For reasons of simplicity, these elements of the device for imaging the interior of a turbid medium which are known in the art will not be described again.

The receiving portion 2 is further structured such that a space remains between the inner surface of the receiving portion 2 and the turbid medium 1. For examination, this space is filled with an optically matching medium. The optically matching medium (in some cases also designated as coupling medium or scattering medium) is selected to provide appropriate optical coupling between the turbid medium 1 to be imaged and the source and detection positions distributed on the inner surface. For this purpose, the optically matching medium is provided with optical properties similar to the optical properties of the turbid medium 1 to be examined.

Now, operation of the device according to the embodiment will be described. The device is particularly adapted for a method in which a turbid medium 1 to which a contrast agent has been administered is examined. The contrast agent is a fluorescent contrast agent capable of emitting (fluorescence) light in a range of wavelengths upon irradiation with suitable light. For example, typical fluorescent dyes used for optical fluorescence tomography emit light in wavelength bands comprising a spectrum from approximately 700-950 nm.

According to the embodiment, first a reference measurement is performed. For this reference measurement, the measurement volume 4 is completely filled with the optically matching medium and the turbid medium is not placed in the measurement volume. In the reference measurement, light from the light source unit 6 is subsequently directed to the interior of the measurement volume 4 from the plurality of source positions. Attenuation of the light used for irradiating which is caused by the optically matching medium is detected in the plurality of detection positions for each source position as reference measurement data, i.e. the attenuation of the incident light by the optically matching medium (acting as a reference medium) is measured. Such a reference measurement need not necessarily always be performed before another turbid medium is examined. For example, a reference measurement can be performed from time to time and the results can be stored in a memory in the control and reconstruction unit 8. The results from such a reference measurement can then be used as a reference for a plurality of different turbid media to be examined.

For examining the interior of a turbid medium, the turbid medium 1 to which contrast agent has been administered is placed in the measurement volume 4 and the remaining space inside the measurement volume 4 is filled with the optically matching medium.

Then, an attenuation measurement is performed in which light from the light source unit 6 is subsequently directed to the turbid medium from the plurality of source positions. In the attenuation measurement, attenuation of the light used for irradiating is detected in the plurality of detection positions for each source position as attenuation measurement data, i.e. the attenuation of the incident light inside the measurement volume 4 is measured, i.e. the attenuation in the turbid medium and the surrounding optically matching medium. Thus, in the attenuation measurement the intrinsic contrast of the turbid medium 1 is used. From these attenuation measurements, a three-dimensional image of the absorption properties of the turbid medium 1 can be reconstructed using reconstruction methods known in the art, as will be described further below.

Further, according to the embodiment, a fluorescence measurement is performed in which the turbid medium 1 is subsequently irradiated with light from the plurality of source positions. The light used in this fluorescence measurement is chosen such that the fluorescent contrast agent (formed by a fluorescent dye preferentially accumulating in lesions in the turbid medium 1) is stimulated by the light to emit fluorescence light comprising wavelengths in a fluorescence range of wavelengths. For example, the irradiation light used for this measurement can comprise the same wavelength (or range of wavelengths) as the irradiation light used for the attenuation measurement. For each of the plurality of source positions, the fluorescence light emitted by the contrast agent is detected in the plurality of detection positions as fluorescence measurement data. Detection of the fluorescence light can e.g. be performed by appropriate filtering in the light paths between the measurement volume 4 and the detection unit 7. Based on the fluorescence measurement data, the control and processing unit 8 reconstructs a volumetric image of the contrast agent distribution in the turbid medium 1, as will be described in the following.

For reconstructing the optical properties of the turbid medium, the results of the attenuation measurement and the results of the reference measurement are exploited. To reconstruct the spatial dependency of the optical properties of the turbid medium, first an initial assumption for the optical properties of the turbid medium to be examined is made. For example, the absorption coefficient μₐ(r,λ) and the diffusion coefficient D(r,λ) are taken into consideration, with r indicating the spatial dependency and λ indicating the wavelength dependency of these optical properties. For example, as an initial assumption the optical properties can be assumed to have no spatial dependency and values corresponding to those of the optically matching medium can be assumed. It should be noted that the method described is not restricted to this and known properties of the turbid medium (such as the outer shape and the like) can already be taken into account for in the initial assumption.

### Step 1)

Based on the initial assumption for the optical properties, initial Green's functions G_{x(s,d)} for the light propagation at the wavelength λₓ of the irradiation light in the measurement volume 4 are calculated (the index s indicating a specific source position, the index d indicating a specific detection position). The Green's functions are calculated as numerical solutions to the diffusion equation with the assumed properties taken into account. For example, the assumed properties may comprise assumed absorption, scattering, geometry, position of the source and position of the detector, boundary conditions, source model, etc. This procedure is well known in the art and e.g. described in Arridge Schweiger, “Photon-measurement density functions. Part 2: Finite-element-method calculations”, Applied Optics, Vol. 34, 8026, 1995.

### Step 2)

In a next step, spatial dependent optical properties μₐ(r,λₓ) (and possibly also D(r,λₓ)) are reconstructed by exploiting the results Φ_{0(s,d)}^{x} from the reference measurement and the results Φ_{(s,d)}^{x} from the attenuation measurement. In this context, Φ_{0(s,d)}^{x} designates the measured intensity at the wavelength λₓ of the irradiation light for the combination of source position s and detector position d with 0 indicating the reference measurement. Analogously, Φ_{(s,d)}^{x} designates the measured intensity at the wavelength λₓ of the irradiation light for the combination of source position s and detector position d of the attenuation measurement. Different schemes for such reconstruction exist depending on the specific type of measurement which is used. According to one example, only μₐ(r,λₓ) is reconstructed. According to a more sophisticated example, D(r,λₓ) is also reconstructed. Linear or non-linear methods for reconstruction can be applied. Linear reconstruction methods are e.g. explained in T. Nielsen, et al. “Linear image reconstruction for a diffuse optical mammography system in non-compressed geometry using scattering fluid”, Applied Optics Vol. 48, 10 D1-D13 (2009). Non-linear reconstruction methods are e.g. explained by Ziegler “Modeling photon transport and reconstruction of optical properties for performance assessment of laser and fluorescence mammography and analysis of clinical data”, FU Berlin, Fakultät für Physik, Doktorarbeit (2008); by Deghani, et. al. “Multiwavelength three-dimensional near-infrared tomography of the breast: initial simulation, phantom and clinical results.”, Applied Optics, Vol. 42, 135 ff, 2003; and by Arridge, “Optical tomography in medical imaging”, Inverse Problems, Vol. 15, R41 ff (1999)

### Step 3

In a next step, the Green's functions G_{x(s,d)}^{k} (in the following also designated as Gₓ^{k}(r_{d},rₛ) for the wavelength λₓ of the irradiation light are calculated again, but now based on the reconstructed optical properties μₐ(r,λₓ) (and possibly also D(r,λₓ)). This is again achieved by calculating the Green's functions as numerical solutions of the diffusion equation as described above.

### Step 4

Further, Green's functions G_{f(s,d)}^{k} (in following also designated as G_{f}^{k}(r_{d},rₛ) for the fluorescence light are calculated based on the reconstructed optical properties by calculating the Green's functions as numerical solutions of the diffusion equation. This is achieved in the same way as described above, but here assumed properties at the fluorescence wavelength are used. For example, in this step the following assumption is made for the optical properties at the fluorescence wavelength: μₐ^{k}(r,λ_{f})=μₐ(r,λₓ)+c_{f}^{k}(r)*(ε(λ_{f})−(ε(λₓ)) and D^{k}(r,λ_{f})=D^{k}(r,λₓ). In this context, k is used as an index which will be iteratively increased (starting from k=0), as will be explained later. c_{f}^{k}(r) is the spatial dependent concentration of the fluorescent contrast agent; ε(λₓ) is the molar absorption coefficient of the fluorescent contrast agent at the wavelength λₓ of the irradiation light; and ε(λ_{f}) is the molar absorption coefficient of the fluorescent contrast agent at the wavelength λ_{f} of the fluorescence light. Since the spatial dependency of the concentration of the fluorescent contrast agent will not yet be known in this step, c_{f}⁰(r) can be set to 0 in this step 4, for example.

### Step 5

In a next step, the spatial distribution of the fluorescent contrast agent in the measurement volume 4 (i.e. the spatial distribution of the fluorescent contrast agent in the turbid medium 1) is reconstructed based on the results Φ_{(s,d)}^{f} of the fluorescence measurement and the results Φ_{(s,d)}^{x} of the attenuation measurement. Φ_{(s,d)}^{f} designates the measured intensity at the wavelength λ_{f} of the fluorescence light for the combination of source position s and detector position d. It should be noted that it is only an assumption that the fluorescence light is emitted at a specific wavelength λ_{f}. In reality, fluorescence light will be emitted in a band of wavelengths. However, for typical contrast agent concentrations this assumption can be made without causing any problems. For example, the wavelength of the fluorescence band of wavelengths at which the fluorescence emission has its maximum can be chosen as λ_{f} in this case.

According to the embodiment, the spatial distribution of the concentration of the fluorescent contrast agent c_{f}^{k+1}(r) is reconstructed by inverting the following equation:

$$\frac{{Φ}_{( s , d )}^{f}}{{Φ}_{( s , d )}^{x}} = C \int {c}_{f}^{k + 1} ( r ) \frac{{G}_{f}^{k} ( {r}_{d} , r ) {G}_{x}^{k} ( r , {r}_{s} )}{{G}_{x}^{k} ( {r}_{d} , {r}_{s} )} {d}^{3} r$$

Here, C is a constant factor which depends on contrast agent properties. Thus, a reconstructed spatial distribution of the fluorescent contrast agent is achieved as a result. When step 5 is executed for the first time, the spatial distribution of the contrast agent concentration will be c_{f}¹(r) with the index 1 indicating the first reconstruction.

### Step 6

In a next step, the reconstructed spatial distribution of the concentration of the fluorescent contrast agent c_{f}^{k+1}(r) is fed back to Step 4. I.e. the Green's functions G_{f(s,d)}^{k+1} for the fluorescence light are recalculated based on the reconstructed optical properties and based on the fed back fluorescent contrast agent concentration by calculating the Green's functions as numerical solutions of the diffusion equation. This is achieved in the same way as described above with respect to Step 4, but now the following is calculated (i.e. k being increased by 1): μₐ^{k+1}(r,λ_{f})=μₐ(r,λₓ)+c_{f}^{k+1}(r)*(ε(λ_{f})−ε(λₓ)) and D^{k+1}(r,λ_{f})=D^{k+1}(r,λₓ). In other words, the contrast agent concentration c_{f}^{k+1}(r) calculated in Step 5 is used as an input for recalculating the Green's functions.

### Step 7

In a next step, the spatial distribution of the concentration of the fluorescent contrast agent is reconstructed again (now c_{f}^{k+2}(r)) in the same manner as in Step 5 but now based on the recalculated Green's functions obtained in Step 6. As a consequence, improved results for the spatial distribution of the concentration of the fluorescent contrast agent are achieved as compared to the first reconstruction obtained in Step 5.

### Further Iterations

In further iterations, the method described with respect to Steps 4 to 7 is repeated, each time feeding back the reconstructed spatial distribution of the contrast agent concentration to the step for calculating the Green's functions at the fluorescence wavelength and redoing the reconstruction of the spatial distribution of the contrast agent reconstruction based on the updated Green's functions. The iterations are stopped when a convergence criterion is met (which can be pre-defined e.g. in a memory in the control and reconstruction unit 8) or when an operator stops the iterative recalculation process.

Since the reconstruction results are iteratively fed back, the quality of a reconstructed fluorescence image is iteratively improved and improved results can be achieved as compared to fluorescence reconstruction without such a feedback loop.

It should be noted that the device for reconstructing a fluorescence image of the interior of a turbid medium according to the embodiment is adapted such that the control and reconstruction unit 8 controls operation of the device such that all the steps described above are performed in an automated manner. This can e.g. be realized by a suitable program implemented in a memory of the device.

Thus, according to the embodiment an iterative reconstruction algorithm is provided that solves the coupled differential equations for irradiation light and fluorescence light by taking the absorption of the fluorescent contrast agent into account. This is achieved by applying a feedback loop using the reconstruction results of a former reconstruction step. The reconstructed concentration of the fluorescent contrast agent which is e.g. calculated from a Born approximation is used to calculate new Green's functions describing the propagation of light but now taking the influence of the reconstructed contrast agent concentration into account.

In one step, the Green's functions for the propagation of irradiation light and fluorescence light are calculated by solving the diffusion equation using optical properties calculated e.g. by linear (Rytov-approximation) calculation or non-linear calculation. The results from this step are used in a next step to reconstruct the spatial distribution of the contrast agent concentration (e.g. using the Born approximation). Since the concentration of the contrast agent has not been known before, self-absorption has not been taken into account so far. Thus, in this step an approximated spatial distribution of the contrast agent concentration is achieved. By feeding back the approximated spatial distribution of the contrast agent concentration to the step of calculating the Green's functions, the knowledge of the (approximated) spatial distribution of the contrast agent concentration helps improving the Green's functions such that self-absorption of the contrast agent can be taken into account. By cycling this loop several times, the knowledge of the spatial distribution of the contrast agent concentration is stepwise improved and the effects of self-absorption are incorporated more correctly into the calculation with every iteration step.

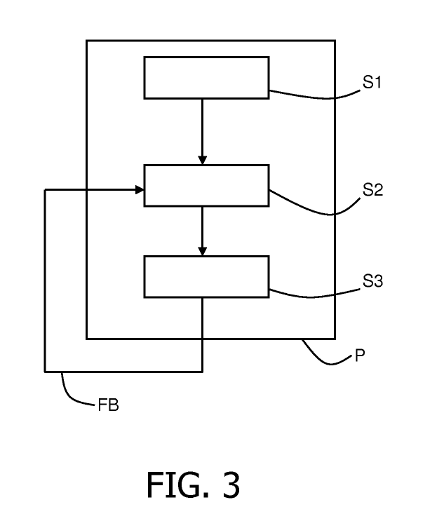

This scheme is schematically shown in FIG. 3. In a step S1, an initial assumption about optical properties of the turbid medium is made. In a step S2, the Green's functions are calculated based on the initial assumption. In a step S3, the spatial distribution of the contrast agent concentration is reconstructed. Thus far, the method corresponds to the scheme known to the applicant before (the steps shown in box P). However, according to the embodiment the feedback loop FB is provided with which the results from the reconstruction process in step S3 are fed back to the step of calculating the Green's functions such that self-absorption of the fluorescent dye is taken into account in the calculations.

### Modification

A modification of the embodiment described above will now be briefly described. According to the modification, attenuation measurements are performed at different wavelengths of the irradiation light. The attenuation of the light used for irradiating is measured for a plurality of different wavelengths λ₁, . . . , λₙ of the light from the light source unit 6. According to the modification, this is accomplished by several different light sources in the light source unit 6, the light sources emitting light at different wavelengths λ₁, . . . , λ₁. Preferably, the light sources are formed by lasers emitting monochromatic light at different wavelengths such as e.g. 690 nm and 730 nm.

From these attenuation measurements, a three-dimensional image of the optical properties of the turbid medium 1 can be reconstructed using the reconstruction methods known in the art and mentioned above. It is possible to use a model to describe the wavelength dependency of the optical properties. This model uses parameters to describe the spatial dependency of the optical properties. It has been shown that it is advantageous to reconstruct model parameters from the attenuation measurements at different wavelengths and then calculate the optical properties from the reconstructed model parameters instead of directly reconstructing the optical properties at the different wavelengths. Several models can be chosen to describe the optical properties. One particular model is described in more detail in the following:

The absorption coefficient of turbid medium 1 may be modeled as a linear combination of a plurality of substances. In other words, the absorption coefficient μₐ is considered to have the following structure:

$${μ}_{a} ( λ , r ) = {\sum }_{i} {c}_{i} ( r ) \cdot {μ}_{ai} ( λ ) ,$$

with cᵢ being the concentration of constituent i, μₐᵢ(λ) being the (wavelength dependent) molar absorption coefficient of constituent i, r being the position in the turbid medium, and the summation being performed over all considered constituents i.

The concentration c_{f}(r) of the fluorescent contrast agent and its absorption coefficient μ_{af} are also taken into account for in the equation

$${μ}_{a} ( λ , r ) = {\sum }_{i} {c}_{i} ( r ) \cdot {μ}_{ai} ( λ , r ) ,$$

i.e. the term c_{f}(r)μ_{af}(λ, r) is taken into account in the summation.

The scattering coefficient of turbid medium 1 may be modeled by:

$${μ}_{s}^{′} ( λ , r ) = A ( r ) \cdot {( \frac{{λ}_{0}}{λ} )}^{b ( r )} ,$$

where the spatially varying parameters A and b are known as scattering amplitude and scattering power, respectively. Using a scattering model is particularly useful if modulated or pulsed measurements are performed.

Based on the data from the attenuation measurements, the local combination of the plurality of constituents is reconstructed for each location in the turbid medium 1. This is done making use of the following equation:

$$ln ( \frac{{Φ}_{x ( s , d )}}{{Φ}_{0 ( s , d )}} ) ∝ {\sum }_{i} {ɛ}_{i} ( {λ}_{X} ) \int d {r}^{3} {c}_{i} ( r ) \frac{{G}_{x} ( {r}_{s} , r ) {G}_{x} ( {r}_{d} , r )}{{G}_{x} ( {r}_{s} , {r}_{d} )} ,$$

wherein i defines the constituents of the turbid medium (such as oxy- and deoxy-hemoglobin, lipid, water, the contrast agent etc.), ε is the molar absorption coefficient for each of these constituents, cᵢ is the respective concentration of constituent i, rₛ defines the source position, r_{d} defines the detection position, r defines the respective position in the turbid medium, and Gₓ defines the Green's functions at wavelength λₓ (with λₓε[λ₁, . . . , λₖ]).

Constituents for which the local concentration can be determined according to this method comprise blood, Hb (oxy-hemoglobin or deoxy-hemoglobin), water, lipid, etc. Thus, the absorption properties of the turbid medium 1 are determined from the attenuation measurements for several discrete wavelengths. Further, the concentration of the fluorescent contrast agent is already (pre-) calculated from the attenuation measurements.

In a next step, in modification to the embodiment described above, according to the modification the Green's functions in Step 3 and Step 4 are calculated exploiting the assumption

$${μ}_{a} ( λ , r ) = {\sum }_{i} {c}_{i} ( r ) \cdot {μ}_{ai} ( λ , r )$$

with the values for cᵢ(r) which have been reconstructed from the attenuation measurements at different wavelengths (and known molar absorption coefficients). In other words, Steps 3 and 4 are performed in a similar manner but using the differently determined μₐ(λ, r) and the pre-calculated c_{f}(r) as c_{f}⁰(r). The following steps and the iterations are performed similar to the embodiment described above.

As a consequence, improved input information for the first reconstruction of the spatial distribution of the contrast agent concentration in Step 5 is provided according to the modification. Thus, even better reconstruction results can be achieved.

Although it has been described above with respect to the embodiments that the attenuation measurements are performed before the fluorescence measurement, a skilled person will understand that the order can be reversed or interleaved or that the measurements can be carried out simultaneously if the detectors are designed in such a way that they can separate fluorescence light from the light of the light source module.

In the embodiments described above, the detection unit comprises a plurality of different detectors the respective detection positions of which are distributed around the measurement volume. This enables fast and efficient measurements. However, different realizations of a plurality of different light paths through the measurement volume are possible as well.

Further, it has been described above that the measurement volume is bounded by a receiving portion having a cup-like shape. However, the measurement volume may also have a different shape, e.g. may be bounded by two parallel plates between which the turbid medium is accommodated in a compressed state during the measurements.

1. Method for reconstructing a fluorescence image of the interior of a turbid medium (1), the method comprising the steps:
accommodating a turbid medium (1) to which a fluorescent contrast agent has been administered in a measurement volume (4),
performing an attenuation measurement by irradiating the turbid medium (1) with irradiation light and detecting light emanating from the turbid medium;
performing a fluorescence measurement by irradiating the turbid medium (1) with light causing the fluorescent contrast agent to emit fluorescence light and detecting the fluorescence light emanating from the turbid medium;
the method further comprising the steps:
i) calculating initial Green's functions for the light propagation in the turbid medium for irradiation light from the diffusion equation based on an initial assumption for the optical properties of the turbid medium;
ii) reconstructing optical properties as a function of the position in the interior of the turbid medium based on the results of the attenuation measurement;
iii) calculating updated Green's functions for the light propagation in the turbid medium for irradiation light from the diffusion equation based on the reconstructed optical properties of the turbid medium;
iv) calculating updated Green's functions for the light propagation in the turbid medium for the fluorescence light from the diffusion equation;
v) reconstructing the spatial distribution of the fluorescent contrast agent based on the results of the fluorescence measurement;
vi) recalculating updated Green's functions for the light propagation in the turbid medium for fluorescence light from the diffusion equation based on the reconstructed optical properties of the turbid medium and based on the reconstructed spatial distribution of the contrast agent; and
vii) reconstructing an updated spatial distribution of the fluorescent contrast agent based on the results of the fluorescence measurement.

2. Method according to claim 1, wherein the results of the attenuation measurement and the results of the fluorescence measurement each comprise values for a plurality of different light paths through the interior of the measurement volume.

3. Method according to claim 1, wherein, in step ii), the optical properties of the turbid medium (1) are reconstructed based on the results of the attenuation measurement and on the results of a reference measurement with the measurement volume (4) filled with a reference medium.

4. Method according to any one of claims 1 to 3, wherein, in steps v) and vii), the spatial distribution of the fluorescent contrast agent inside the turbid medium (1) is reconstructed based on the results of the fluorescence measurement and on the results of the attenuation measurement.

5. Method according to claim 1, wherein, in step vii), the updated spatial distribution of the fluorescent contrast agent inside the turbid medium (1) is reconstructed exploiting the updated Green's functions for the light propagation in the turbid medium for fluorescence light recalculated in step vi).

6. Method according to claim 1, wherein steps vi) and vii) are repeated until a convergence criterion is met.

7. Method according to claim 1, wherein attenuation measurements are performed for a plurality of different wavelengths of the irradiation light, and, in step ii), the wavelength dependency of the optical properties is described by a model and the spatial distribution of the model parameters is reconstructed based on the results of the attenuation measurements at the plurality of different wavelengths, and optical properties as a function of the wavelength and position in the interior of the turbid medium are calculated based on the reconstructed spatial distribution of the model parameters.

8. Method according to claim 7, wherein the optical properties are individually calculated for irradiation light and for fluorescence light.

9. Device for reconstructing a fluorescence image of the interior of a turbid medium (1), the device comprising:
a measurement volume (4) adapted for receiving a turbid medium (1) to be examined;
a light source unit (6) adapted for irradiating the interior of the measurement volume (4);
a detection unit (7) adapted for detecting light emanating from the interior of the measurement volume (4); the light source unit (6) and the detection unit (7) being adapted such that a plurality of combinations of source position and detection position corresponding to a plurality of different light paths through the measurement volume (4) are provided; and
a control and reconstruction unit (8) adapted to control the device to:
perform an attenuation measurement by irradiating the turbid medium with irradiation light and detecting light emanating from the turbid medium;
perform a fluorescence measurement by irradiating the turbid medium with light causing a fluorescent contrast agent administered to the turbid medium to emit fluorescence light and detecting the fluorescence light emanating from the turbid medium;
the control and reconstruction unit (8) being adapted to:
i) calculate initial Green's functions for the light propagation in the turbid medium for irradiation light from the diffusion equation based on an initial assumption for the optical properties of the turbid medium;
ii) reconstruct optical properties as a function of the position in the interior of the turbid medium based on the results of the attenuation measurements;
iii) calculate updated Green's functions for the light propagation in the turbid medium for irradiation light from the diffusion equation based on the reconstructed optical properties of the turbid medium;
iv) calculate updated Green's functions for the light propagation in the turbid medium for the fluorescence light from the diffusion equation;
v) reconstruct the spatial distribution of the fluorescent contrast agent based on the results of the fluorescence measurement;
vi) recalculate updated Green's functions for the light propagation in the turbid medium for fluorescence light from the diffusion equation based on the reconstructed optical properties of the turbid medium and based on the reconstructed spatial distribution of the contrast agent; and
vii) reconstruct an updated spatial distribution of the fluorescent contrast agent based on the results of the fluorescence measurement.

10. The device according to claim 9, wherein the light source unit (6) is adapted to selectively irradiate the interior of the measurement volume (4) with light at a plurality of different wavelengths (λᵢ).

11. The device according to claim 9, wherein the control and reconstruction unit (8) is adapted to iteratively repeat steps vi) and vii).

12. The device according to claim 9, wherein the device is a medical image acquisition device.
